(19) United States
(12) Patent Application Publication
(10) Pub. No.: US 2003/0124618 A1
(43) Pub. Date: Jul. 3, 2003

(54) Device for analysing analyte compounds and use hereof

(75) Inventors: Fei Chen, Lynge (DK); Ann Merete Moller, Bagsvaerd (DK)

Correspondence Address: HUNTON & WILLIAMS, INTELLECTUAL PROPERTY DEPARTMENT, 1900 K STREET, N.W., SUITE 1200, WASHINGTON, DC, 20006-1109, US

(73) Assignee: Lattec I/S, Hillerod (DK)

(21) Appl. No.: 10/309,053

(22) Filed: Dec. 4, 2002

Related U.S. Application Data
(60) Provisional application No. 60/334,941, filed on Dec. 4, 2001.

(51) Int. Cl. G01N 33/53; C12M 1/34; G01N 33/558

(52) U.S. Cl. 435/7.1; 435/287.2; 436/514

(57) ABSTRACT

A device for assaying a steroid compound in a liquid sample is provided. The device comprises a porous zone permitting the migration of the liquid sample. The said porous zone comprises a first zone, a second zone and a third zone. The first zone comprising a first non-immobilised conjugate, capable of specifically binding to the steroid to be assayed and a second non-immobilised conjugate capable of binding specifically to a compound different from the steroid to be assayed. The second zone comprises the same type of steroid as the one to be assayed which is being immobilised to the porous zone. The third zone comprising the second member of the reference pair to the second conjugate.

### FIELD OF INVENTION

The present invention relates in general to the field of analysing analytes in biological samples. In particular, a novel device for competitive immunoassay for assaying steroid compounds in such samples including milk samples is provided with the objective of improving reproductive performance of dairy herds.

### TECHNICAL BACKGROUND AND PRIOR ART

One of the major factors affecting the profitability of dairy enterprises is the calving interval. A calving interval of 365 days is ideal. Poor reproductive performance results in longer calving intervals which evidently have a negative impact on profitability. The variability in the economic impact of reproductive performance can be inferred from the marked differences between selected countries in respect of conception rates relative to first insemination. In New Zealand for example, where calving according to season is important, an average conception rate of 85% has been reported. In Great Britain, where milk production from grazed grass is less financially important, the average conception rate is currently down to around 40%.

In the last decade there has been a dramatic decline in the average reproductive performance in dairy herds. The interval between calvings has increased by 20 days and the number of cows conceiving after first insemination has dropped by about 15%. Although there have been substantial changes in dairy management over this period such as increases in herd size and increasing automation, it is implausible that the decline in reproductive performance can be ascribed solely to a universal worsening in the reproductive management ability of dairy farmers. Over a similar time span, it has been shown that genetic selection for milk yield has resulted in decreased reproductive performance at the genetic level. There is a strong association between milk production potential of the cows, body energy mobilisation and decreased reproductive performance. Thus current poor reproductive performance is not only costly but it is also on the increase.

The key factors for achieving an optimal calving interval include the length of post-calving anoestrus, precise oestrus detection and rapid follow up of cows not conceiving at the first insemination. Biologically speaking, an extended post-calving anoestrus period indicates that the cow is experiencing metabolic stress in early lactation, typically mobilising excessive amounts of body energy reserves. Other stress factors such as disease and social competition can also affect the length of the post-calving anoestrus. However, in the absence of physiological measures extended anoestrus periods may merely reflect a failure to detect first oestrus. Once the cow is cycling the interval from first oestrus to conception is largely determined by reproductive management skill.

Precise detection of oestrus and thus correct timing of insemination has a substantial effect on subsequent conception rate. For the cows that do not conceive, the earliest possible recognition hereof reduces the delay in getting those particular cows bred again.

Currently, determining the point in time during oestrus, where insemination is most likely to result in conception, is generally based on visual inspection of heat manifestations which may give rate and accuracy of oestrus detection in excess of 90%. However, this is possible only when skilled personnel is used and only if a substantial amount of time is devoted to this activity. Accordingly, reliable objective physiological indicators of reproductive status would be of considerable value. Given examples are: milk temperature profiles have been suggested, physical activity tags or combined observations of temperatures and physical activity are such indicators. However, none of these indicators are entirely reliable indicators of oestrus.

A further significant prerequisite for securing optimal reproductive performance of a dairy herd is the ability to determine as early as possible after insemination that conception has been achieved. An unsuccessful conception must be followed up by repeated insemination.

Detection of pregnancy in cows is currently made by rectal palpation but this method is only reliable when applied 5 to 6 weeks after insemination. In most cases cows that have failed to conceive are defined as those showing oestrus signs about 21 or 42 days after insemination. However, this approach relies upon good oestrus detection skills. It has been reported that re-insemination of already pregnant cows may occur in up to 20% of second inseminations. This is both an unnecessary cost and involves a risk of inducing abortion of the original conceptus. An objective and reliable early measure of pregnancy would thus be a useful reproductive management aid.

There are a number of studies that have attempted to measure molecules that are pregnancy specific, such as oestrone sulphate (only after day 45), pregnancy specific proteins and pregnancy-associated glycoproteins. However, interpretation of these measures has not yet proved to be entirely straightforward, partly because there can be many different aetiologies of early embryo loss. A more promising alternative is β-mode ultrasonography, whereby foetal heart beat has been detected as early as 20 days after conception.

Several attempts have been made to identify compounds indicative of the state in the reproductive cycle. The detection of such compounds in milk might be reliable indicators of reproductive performance, including oestrus detection and detection of conception. One such compound, whose usefulness for these purposes has been investigated, is progesterone. Progesterone is a steroid hormone that is mainly produced by the corpus luteum and to some extent the placenta of the pregnant cow. It is found in ng/ml concentrations in plasma. There is a close correlation between plasma progesterone and milk progesterone (r=0.90; Abeyawandene et al., 1984). Progesterone being a steroid hormone, is fat soluble and is consequently found in different concentrations in defatted milk and milk fat. Defatted milk concentrations of progesterone are approximately 50% of plasma concentrations. Milk fat concentrations of progesterone are 5 to 10 times higher than plasma concentrations.

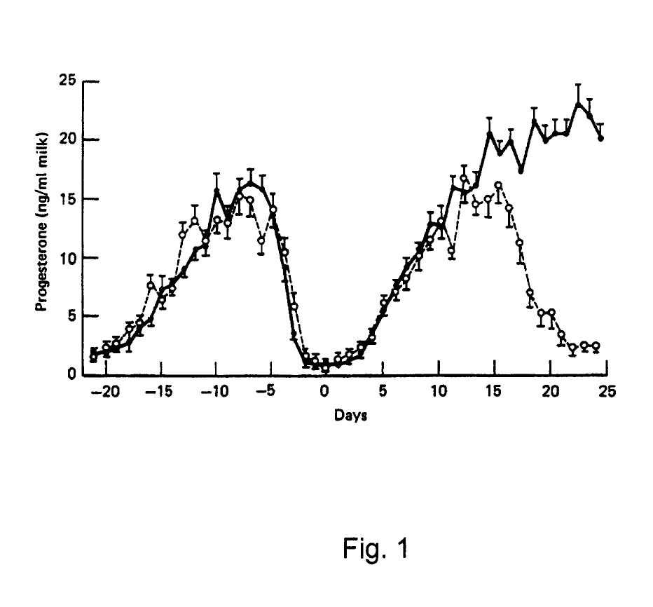

Whole milk progesterone concentrations are between those of defatted milk and milk fat and reflect the relative proportions of these two milk fractions. Consequently, milk progesterone concentrations have been found to vary with milk fat content (Waldmann, 1993). A typical pattern of whole milk progesterone throughout one oestrus cycle and the onset of a subsequent pregnancy is shown in FIG. 1. The important feature is the large difference between progesterone concentrations in the luteal phase (days −16 to −4) and progesterone in the period around oestrus (days −3 to +3). Detecting this difference may form the basis not only for oestrus detection but also pregnancy detection and detection of length of the postpartum anoestrus.

Pregnancy detection, using progesterone would rely upon differentiating between the normal progesterone pattern of a non-pregnant cycling cow and the pattern of the pregnant cow. As can be seen in FIG. 1, this could, in theory, occur as from approximately 15 days after a correctly timed insemination. The progesterone level of the cycling cow starts to decline as she comes into oestrus, whilst that of the pregnant cow remains high. The necessary precision to distinguish pregnant from non-pregnant cows, in an individual progesterone determination decreases with increasing days from insemination until the progesterone pattern of the following cow cycle starts raising again, at about 24 days after oestrus. However, the main purpose of progesterone measurements after insemination is to detect non-pregnant cows which require re-insemination. Most literature reports indicate that reliable differentiation of pregnant from non-pregnant cows occurs around day 20 after conception.

Oestrus periods can be reliably identified from progesterone patterns, where samples are taken every second or third day (Royal et al., 2000). However, progesterone levels are low throughout the period from 2-3 days before until 2-3 days after oestrus, and successful insemination depends upon precise identification of oestrus itself. Salisbury and VanDemark (1961) showed that timing of insemination relative to oestrus, had a dramatic effect on pregnancy rates. The optimum time for insemination is 4-8 hours after ovulation. Furthermore, individual cows show considerable variation, in both the time interval between the drop in progesterone levels and the onset of oestrus as well as in the duration of oestrus itself.

Generally, conventional assays for detecting analytes in biological samples include competitive immunoassays and immunoassays based on the “sandwich” technique.

Competitive immunoassays are generally based on designs, where an antibody against the analyte is immobilised on a solid support and labelled analyte is added to the sample. If non-labelled analyte is present in the sample, this native analyte and the labelled analyte will compete for binding sites on the immobilised antibody. This approach however is associated with several problems: it is technically difficult to label relatively small analytes. The presence of a relatively large labelling substance may result in steric hindrance of the steroid-antibody binding for instance, in turn resulting in a less than optimal sensitivity of the assay.

The above problem, arising from poor coupling performance in such conventional competitive immunoassays, may be overcome by using a “sandwich” type immunoassay. In such an assay format, an antibody against the analyte to be analysed is immobilised on a solid support and a labelled antibody against the analyte is applied to the sample. The analyte is bound between the two antibodies. One problem associated with “sandwich” immunoassays is that this assay format is most suited for the detection of analytes that have at least two antigenic sites. Additionally, this type of immunoassays requires a relatively high amount of antibodies and therefore becomes more expensive.

EP 0 282 192 discloses a type of competitive immunoassay for qualitative detection of a hapten in milk. The assay is based on adding to the milk sample a labelled antibody against the hapten to be assayed and a labelled substance capable of binding to a binding partner which is different from the hapten. The milk sample is analysed by applying to it a solid support provided with two zones, one zone to which molecules of the hapten to be assayed are immobilised and another to which the different binding partner is immobilised (reference zone). In the presence of the hapten in the milk, a relatively weak label signal is generated relative to the signal from the reference zone. This assay format is very laborious because it requires that the user performs very precise measurements of additional reagents during use. Additionally, this assay format does not permit reproducible and reliable quantitative measurements of small amounts of haptens such as steroids, which is due to the disadvantage that the assay device is applied to the liquid sample rather than applying the liquid sample to the assay device.

Other examples of immunoassays that may be used as true-false tests or tests for semi-quantitative detection of steroids in liquid samples are disclosed in EP 0 325 449 and EP 0 810 436.

EP 1 248 111 discloses a kit and a method for detecting progesterone in a test sample such as milk. The kit comprises a porous membrane having a test line in which progesterone has been immobilised. When performing the assay, the test sample is mixed with an anti-progesterone IgG-gold conjugate before applied to the porous membrane. One of the disadvantages of this assay is that it introduces additional risks due to the mixing step and needs very precise measurements of the additional reagent during use. This handling of reagents also requires that the assay is performed by trained personal. Thus, this assay is not very sensitive or reproducible and therefore, not usable for the quantitatively determination of small day to day changes.

WO 01/50129 discloses a method for quantitatively determining an analyte, preferably a protein or a peptide compounds, in a fluid sample. The method uses a membrane strip comprising an application point, a contact region and a detection zone. The contact region having 1) a population of test particles imbedded therein, wherein the test particles are coated with an analyte binding agent, and 2) a population of internal control particles imbedded therein, wherein the internal control particles are coated with a control analyte binding agent. The detection zone comprises an immobilised detection reagent capable of binding to contacted test particles which are insufficiently coated with the analyte of interest. In order to obtain a sufficient sensitivity of the assay, the non-specifically binding within the detection zone is determined from the amount of internal control particles in the detection zone. The provision of this determination requires that the labels of the test particles and the internal control particles are distinguishable. Thus, due to e.g. the distinguishable labels the assay becomes expensive and the requirements to the equipment are more extensive and the determination is more complex.

Other types of immunoassays describing a device for semi-quantitative or quantitative detection of an analyte present in a liquid sample wherein a molecule which binds specifically to the analyte to be detected (such as an antibody) is immobilised in a detection zone of the device are disclosed in US 2001/026944 and WO 00/31538.

As described above measurements of progesterone in milk samples are potentially useful means of optimising reproductive performance, provided analytical methods are available that permit reproducible detection of very small quantities of the steroid, that is in the range of 0-50 ng/ml. Thus, it is a requirement for such an assay that it allows very small day-to-day changes in the level of progesterone, particularly around oestrus and around the point in time where pregnancy and non-pregnancy levels begin to deviate, to be detected.

Accordingly, a suitable assay for monitoring progesterone levels on a daily basis or during selected time intervals must be cheap, highly sensitive, reproducible and easy to perform and preferably be compatible with automated and semi-automated systems for optimising performances of milk producing herds, such as the system disclosed in co-pending European patent application No. 01610022.4. Such an analytical method and the means for performing the method are provided herein.

### SUMMARY OF THE INVENTION

Accordingly the present invention provides a device and a method for quantitative determination of an analyte in a liquid sample such as milk. The present invention makes it possible to monitor an analyte of interest on a daily basis or during selected time intervals in a way which is cheap, highly sensitive, reproducible and easy to perform.

The device according to the present invention permits the liquid sample to migrate through a first zone and thereby releasing at least two different specifically binding conjugates from the first zone. A first specifically binding conjugate that is able to specifically bind the analyte of the sample and a second specifically binding conjugate designed not to react with the analyte or the first specifically binding conjugate. Upon the release of the specific conjugates, the liquid sample now containing the specifically binding conjugates migrates through a porous zone towards a second zone and further on to a third zone. When passing the second zone, any non-bound first specifically binding conjugate in the liquid sample is bound to molecules similar to the analyte or analogue thereof which have been immobilised in the second zone. Excess of first specifically binding conjugate is thereby bound and the amount of this excess of first specifically binding conjugate, is rendered measurable. In the third zone, any non-bound second specifically binding conjugate in the liquid sample is bound to molecules not reactive with the first specifically binding conjugate or the analyte or derivatives of the analyte which have been immobilised in this third zone. The bound second specifically binding conjugate is hereby rendered measurable and serves-as the internal reference which enables the quantitative determination of the analyte.

The quantitative determination of the analyte in the liquid sample is enabled by using the second specifically binding conjugate as an internal reference for the migration rate of the first specifically binding conjugate, as both conjugates will have the same relative migration rate through the first zone and through the porous zone, enabling a calibration for differences in migration rates due to differences in structure of the porous zone.

### DETAILED DISCLOSURE OF THE INVENTION

Thus, in the broadest aspect of the present invention, a device and a method for assaying an analyte in a liquid sample is provided wherein said device comprises a porous zone permitting the migration of the liquid sample. The device comprises:

1. a first zone comprising

a non-immobilised first specifically binding conjugate consisting of a labelled first molecule capable of specifically binding to the analyte to be assayed, wherein the label is detectable by appropriate means,

a non-immobilised second specifically binding conjugate consisting of a labelled second molecule capable of binding specifically to a compound different from the analyte to be assayed, the described second conjugate is not capable of specifically binding to the analyte to be assayed nor to the first specifically binding conjugate,

2. a second zone comprising

an immobilised molecule similar to the analyte to be assayed, or

an analogue thereof this same type of analyte or analogue thereof is capable of specifically binding the first specifically binding conjugate, and

3. a third zone comprising immobilised thereto said compound different from the analyte to be assayed, that is capable of binding specifically to the second specifically binding conjugate.

Under assaying conditions a known amount of the liquid sample is added to an application zone which is connected to the porous zone. The liquid sample is permitted to migrate through the first zone towards the porous zone in which the liquid sample and the first specifically binding conjugate and the second specifically binding conjugate migrate towards and through the second zone as well as the third zone. Any unbound material is absorbed in an absorbent zone that is located downstream of the third zone and provides a force to the liquid sample to direct the migration through the first zone, the second zone and the third zone. The quantitative determination of the molecule to be assayed can be determined by a relationship between the signal intensities, given by the labels specifically bound to the second zone and the third zone. This provides, in accordance with the present invention, a suitable assay for monitoring small molecule levels for instance on a daily basis or during selected time intervals in a cheap, highly sensitive, reproducible and easy manner.

In accordance with the present invention, the device comprises a porous zone wherein a liquid sample can migrate. In this porous zone the first specifically binding conjugate, the second specifically binding conjugate, the analyte and other non-analyte compounds present in the applied liquid sample are migrating together in the liquid sample. In the present context the term “migrate” relates to the movement of the liquid sample through the porous zone. This movement of the liquid sample can be provided by actions such as floating action, capillary action or gravity action. The migration proceeds when the porous membrane is made wet for instance by the liquid sample.

In the present context, the term “specifically binding” relates to the binding between a pair of molecules (each being a member of a specifically binding pair) which are naturally derived or synthetically produced. One of the pair of molecules has an area on its surface, or a cavity which specifically binds to, and is therefore defined as complementary with a particular spatial and polar organisation of the other molecule, so that the pair has the property of binding specifically to each other. Examples of types of specifically binding pairs are antigen-antibody, biotin-avidin, hormone-hormone receptor, receptor-ligand, enzyme-substrate, IgG-protein A.

In a preferred embodiment of the present invention, the porous zone comprises a porous material that absorbs the liquid sample and thereby permits it to migrate. The porous material selected comprises a poresize and has a capacity that makes it possible to provide a high flow-rate that reduces the assay time. Preferably, the porous zone is selected from a porous material providing substantially no retention of triglyceride rich samples. Said porous material is preferably selected from the group consisting of a nitrocellulose membrane, a polymer such as nylon, polyvinylidene fluoride or latex, glass fibre, woven fibres, non-woven fibres and a chromatographic gel membrane. Furthermore, the porous zone provides the connection between the first zone, the second zone, the third zone and the absorbent zone. In addition, the porous zone is also used as the basis for immobilisation in the second zone of the same type of analyte as the one to be assayed or an analogue thereof and for immobilisation in the third zone of a molecule not reactive to the analyte to be assayed or the first specifically binding conjugate and which molecule is capable of binding specifically to the second specifically binding conjugate.

In a preferred embodiment of the present invention, the porous zone comprises a porous material comprising a suitable pore size such as at most 500 μm, for instance at most 200 μm, such as at most 150 μm, for instance at most 100 μm, such as at most 75 μm.

In another preferred embodiment of the present invention, the porous zone comprises a porous material comprising a pore size preferably in the range of 10-30.000 μm, such as in the range of 10-20.000 μm, for instance in the range of 10-10.000 μm, for instance in the range of 10-1000 μm, such as in the range of 10-500 μm, such as in the range of 10-100 μm, for instance in the range of 10-75 μm, such as in the range of 10-50 μm, for instance in the range of 50-200 μm, such as the range of 50-100 μm, for instance in the range of 100-500 μm, such as the range of 50-300 μm, for instance in the range of 75-300 μm, such as the range of 75-200 μm, for instance in the range of 75-150 μm, such as the range of 75-120 μm.

In another preferred embodiment of the present invention, the porous material is characterised by having a high capacity for binding proteins such as at most 400 μg/cm², for instance at most 250 μg/cm², such as at most 200 μg/cm², for instance at most 140 μg/cm², such as at most 120 μg/cm², for instance at most 100 μg/cm², such as at most 80 μg/cm², for instance at most 60 μg/cm², such as at most 40 μg/cm², or the porous material is characterised by having a high capacity for binding proteins such as in the range of 1-400 μg/cm², for instance the range of 1-250 μg/cm², such as the range of 1-200 μg/cm², for instance the range of 1-140 μg/cm², such as the range of 1-120 μg/cm², for instance the range of 1-100 μg/cm², such as the range of 1-80 μg/cm², for instance the range of 1-60 μg/cm², such as the range of 1-40 μg/cm², for instance the range of 50-200 μg/cm², such as the range of 50-100 μg/cm², for instance the range of 50-150 μg/cm², such as the range of 50-120 μg/cm², for instance the range of 75-120 μg/cm², such as the range of 75-110 μg/cm².

Accordingly, in a preferred embodiment of the present invention, the porous material is characterised in that it permits the liquid sample to migrate with a high capillary flow rate, such as at most 300 sec/4 cm, for instance at most 200 sec/4 cm, such as at most 100 sec/4 cm, such as at most 75 sec/4 cm, or the porous material is characterised in that it permits the liquid sample to migrate with a high capillary flow rate, such as in the range of 50-500 sec/4 cm, for instance in the range of 50-250 sec/4 cm, such as in the range of 50-200 sec/4 cm, such as in the range of 50-100 sec/4 cm, for instance in the range of 50-75 sec/4 cm, such as in the range of 100-250 sec/4 cm, including in the range of 150-250 sec/4 cm, such as in the range of 200-250 sec/4 cm and for instance in the range of 250-500 sec/4 cm, such as in the range of 75-150 sec/4 cm and for instance in the range of 80-130 sec/4 cm, such as in the range of 80-110.

In accordance with the above mentioned pore size, capacity and flow-rate, it is desirable to provide a device that permits the detection of an analyte in a fast assay. Preferably, the assay time is less than 15 minutes, preferably less than 10 minutes, preferably less than 8 minutes, preferably less than 7 minutes, preferably less than 6 minutes, preferably less than 5 minutes, preferably less than 4 minutes, preferably less than 3 minutes, preferably less than 2 minutes, preferably less than 1 minute, preferably less than 30 seconds.

In accordance with the present invention, the second zone and the third zone constitute parts of the porous zone and the material used in these two zones is the same as the material used in the porous zone.

In one preferred embodiment of the present invention, the device is provided with at least one second zone and at least one third zone to provide a device for detecting at least one analyte in a liquid sample. The total number of detectable zones in the device of the present invention is at least 2 zones, such as 3 zones, for instance 4 zones, such as 5 zones, for instance 6 zones, such as 10 zones, for instance 15 zones, such as 30 zones, for instance 50 zones where the number of detectable second zones and detectable third zones are selected according to the need of the number of internal references. Here it is possible, by simultaneous assaying a multiplicity of analytes, to retrieve more analytical data from one liquid sample in one assay run and by the use of a single assaying device.

In another preferred embodiment of the present invention the device is equipped with calibration reference in addition to the internal reference in the third zone. In the present context, the term “calibration reference” relates to a coloured zone added to the porous zone unrelated to any specifically binding reactions. This calibration reference provides the same shade over and over again.

In another embodiment of the present invention, the second and third zones are located in parallel, compared to each other in the device and the application zone is located upstream of both sites.

In another preferred embodiment of the present invention, the second and third zones are placed in parallel, compared to each other in the device and the application zone is located between the two sites. In this preferred embodiment, no additional means are necessary to be supplied to the device for directing the flow of the liquid sample.

It is evident that the device according to the present invention may have all kinds of shapes such as circularly shaped, conically shaped or shaped as a triangle, where at least one second zone and at least one third zone are located around the centre of the device whose centre may act as an application zone.

Another preferred embodiment of the present invention provides an appliance comprising a multiplicity of devices applicable for an automatic, a semi-automatic and a continuous system. This kind of system could be, but is not limited by, the system disclosed in the copending European patent application No. 01610022.4. In the present context, the term “appliance” relates to an apparatus which constantly presents devices according to the invention for the detection of the analyte to be assayed and this provision proceeds without manual operations. The appliance according to the present invention is selected from the group consisting of a strip, a band, a tape and a film.

In order to wet the porous material that is used in the porous zone and which permits migration, a liquid sample is applied. In the present context “a liquid sample” relates to any sample found in the form of liquid, solid or gas and which is liquefied at the time of assaying. Additionally, no handling steps, or at least a minimum of handling steps, of the liquid sample are necessary before applying it to the porous zone which comprises the application zone. In the present context, the term “handling steps” relates to any kind of pre-treatment of the liquid sample before or after it has been applied to the assay device. This pre-treatment comprises separation, filtration, distillation, concentration, inactivation of interfering compounds, centrifugation, heating, fixation, addition of reagents, or chemical treatment.

In a preferred embodiment of the present invention, the liquid sample is collected from any kind of mammal, preferably a mammal selected from the group consisting of herd animals, cows, camels, buffaloes, pigs, horses, deer, sheep, goats, pets, dogs, cats and humans.

In a preferred embodiment of the present invention, the liquid sample can be derived from any desirable source such as physiological fluids. Preferably, this source is selected from the group consisting of milk, blood, serum, plasma, saliva, urine, sweat, ocular lens fluid, cerebral spinal fluid, ascites fluid, mucous fluid, synovial fluid, peritoneal fluid, amniotic fluid or the like.

Besides physiological fluids, other liquid samples such as various water samples, food products and the like can be used. In addition, a solid test sample can be used once it is modified to form a liquid sample, for instance in the form of a solution, a suspension or an emulsion.

The porous zone of the device described in the present application comprises a first zone. In the present context, “a first zone” relates to either an integrated site in the porous zone or a separate site connected to the porous zone. The first zone comprises the non-immobilised first specifically binding conjugate and the non-immobilised second specifically binding conjugate. After the application of the liquid sample, it migrates through the said first zone before migrating through the second zone and the third zone. The material used in the first zone provides a fast, consistent and quantitative release of both the non-immobilised first specifically binding conjugate and the non-immobilised second specifically binding conjugate and provides a low or substantially none retention of the triglyceride and proteins in the liquid samples.

In a further embodiment of the present invention, the first zone is separated from the porous zone. In this embodiment the material used can be the same as the material used in the porous zone or it can be different form the material used in the porous zone. This separation gives the opportunity to provide the first zone with different characteristics than the porous zone, for instance different properties in the binding, different migration properties of the first specifically binding conjugate and the second specifically binding conjugate.

In jet a further embodiment of the present invention, the first zone is part of the porous zone to provide a more homogeneous assaying device for the migration of the liquid sample. In this embodiment the material used is the same as the material used in the porous zone.

Preferably the materials used in the first zone, the second zone, the third zone, the porous zone, the application zone and the absorbent zone are the same in at least 6 of the zones, such as at least 5 of the zones, for instance 4 of the zones, such as at least 3 of the zones, for instance 2 of the zones. Such materials are selected from the group consisting of a nitrocellulose membrane, a polymer such as nylon, polyvinylidene fluoride or latex, glass fibre, woven fibres, non-woven fibres and a chromatographic gel membrane.

In a useful embodiment of the present invention, the material to be used in the application zone is a cellulosic material such as Ahlstrom 8975 or Ahlstrom 8964 from Ahlstrom and/or the material to be used in the first zone is a cellulose material such as Ahlstrom 8975 or Ahlstrom 8964 from Ahlstrom and/or the material to be used in the porous zone is a cellulosic material such as product no. HF090 from Millipore and/or the material to be used in the adsorbent zone is a cellulosic material such as product no. D28 from Whatman or Ahlstrom 222 from Ahlstrom.

Additionally, the porous zone comprises “a second zone” and “a third zone”. In the present context, “a second zone” and “a third zone” relate to two detectable sites both preferably located downstream from the first zone and the second zone and the third zone can be partially overlapping or they can be completely separated. The second zone can be located either upstream or downstream to the third zone. Furthermore, the second zone and the third zone can be part of the porous zone or it can be a separate material connected to the porous zone.

In one preferred embodiment of the present invention, a partial overlapping of the second zone and the third zone is present. Preferably, the two zones have about 1% overlap between the areas of the two zones, about 2% overlap between the areas of the two zones, about 3% 4%, 5%, 10%, 20%, 30%, 40%, 50%, 60%, 70%, 80%, 90%, or about 100% overlap between the areas of the two zones is provided. When the second zone and the third zone are overlapping the label selected to the first specifically binding conjugate is different from the label selected to the second specifically binding conjugate, thereby retrieving different signals.

In another preferred embodiment of the present invention, the second zone has immobilised thereto the same kind of analyte as the one to be assayed or an analogue thereof. In the present context, the term “the same kind of analyte as the one to be assayed” relates to any compound comprising the same features and/or the same structure as the analyte to be assayed and in the present context “an analogue thereof” relates to any molecule or compound capable of performing the same specifically binding as the analyte to be assayed.

In the present context, the term “same features as the analyte to be assayed” relates to any compound capable of providing a specifically binding similar to the binding performed by the analyte to be assayed and any compound having the same physical or chemical features as the analyte to be assayed.

In the present context the term, “same structure as the analyte to be assayed” relates to any compound having the same physical or chemical structure as the analyte to be assayed.

Additionally, in the third zone one member of a reference pair is immobilised. In the present context, “a reference pair” refers to two molecules where the molecules through chemical or physical means specifically bind to each other and the two molecules are capable of binding each other with substantially no interference with either the analyte in the liquid sample, the molecule immobilised in the second zone or the non-immobilised first specifically binding conjugate. Preferably, the reference pair is selected from the group consisting of pairs of specifically binding molecules comprising protein-antibody, antigen-antibody, antibody-antibody, lectin-carbohydrate, hormone-antibody and hormone-receptor all providing a specifically binding symbolised by a line (-).

In a preferred embodiment of the present invention, the antibodies used for the reference pair are either monoclonal antibodies or polyclonal antibodies and is produced in a mammal selected from the group comprising mouse, rats, rabbit, goat, sheep, cow and horse. As an example the reference pair comprise an anti-goat antibody immobilised in the third zone and antibodies from goats in the non-immobilised second specifically binding conjugate in the first zone.

The molecule similar to the analyte or analogue thereof to be immobilised in the second zone and the member of the reference pair to be immobilised in the third zone may be immobilised by direct attachment to the porous zone. Preferably, the immobilisations are performed by using a molecular spacer. In the present context, “a molecular spacer” refers to a molecule or a compound capable of linking a molecule similar to the analyte or analogue thereof to the second zone and linking one of the reference members to the third zone and in this way, the molecular spacer comprises a molecule or a compound capable of extending the distance between the porous zone and the molecule similar to the analyte or analogue thereof or the member of the reference pair to be immobilised and makes it more accessible to react with the first specifically binding conjugate and second specifically binding conjugate, respectively.

In one embodiment of the present invention the molar ratio between the spacer molecule and the analyte or analogue being immobilised and/or the molar ratio between spacer molecule and the compound to be immobilised is e.g. 1:99, such as 25:75, e.g. 50:50, such as 75:25, e.g. 99:1. Preferably the molar ratio between spacer molecule and the analyte or analogue being immobilised and/or the molar ratio between spacer molecule and the compound to be immobilised is within the ratio of 10:1, such as 7.5:1, e.g. 5:1, such as 2.5:1, e.g. 0.5:1.

In a useful embodiment of the present invention, the spacer molecule is selected from the group consisting of proteins, peptides, amino acids and small organic molecules. One useful spacer molecule is bovine serum albumin (BSA).

The first zone comprises non-immobilised first specifically binding conjugate and non-immobilised second specifically binding conjugate. In the present context, the term “non-immobilised first specifically binding conjugate” refers to a conjugate which is freely movable in the moist state. Preferably, the conjugate is substantially non-movable when dry and when moistened the conjugates are released and start migrating. Preferred agents, such as detergents, may be added to improve migration of the conjugates and of the liquid sample but also other releasing systems might be used.

The first specifically binding conjugate comprises a first molecule and a detectable label. In the present context, the term “first molecule” refers to a protein, an antibody, a receptor, an enzyme, a peptide, an amino acid, a hormone, a vitamin, a drug or a combination thereof which when attached to a label provides the non-immobilised first specifically binding conjugate and the first molecule is the part of the non-immobilised first specifically binding conjugate that provides the specifically binding capacity to the analyte to be assayed. Preferably the antibody used in the first specifically binding conjugate is a monoclonal antibody specific binding the analyte to be assayed or an analogue thereof.

In useful embodiments of the present invention the molar ratio between first molecule and the detectable label is in the range of 1:99 respectively, such as in the range of 25:75 respectively, e.g. in the range of 50:50, such as in the range of 75:25 respectively and e.g. in the range of 99:1 respectively.

In the present context, the term “label” refers to any substance which is directly or indirectly attached to the first molecule and which is capable of producing a signal that is detectable by visual or instrumental means such as magnotometer, spectrophotometer, ELISA-reader. Various suitable labels for use in the present invention is selected from the group consisting of chromogens, catalysts, fluorescent compounds, chemiluminescent compounds, radioactive labels, metals, magnetic particles, dye particles, enzymes or substrates, or organic polymer latex particles; liposomes or other vesicles containing signal producing substances and the like.

In a preferred embodiment of the present invention, the label is a metal including a metal selected from the group consisting of gold, titanium and silver, but also other elements like carbon and silicon can be used, where no additional manipulation or at least a minimum of manipulation of the label is required to produce a detectable signal.

Preferably the label of the non-immobilised first specifically binding conjugate is selected from the same group as the label of the non-immobilised second specifically binding conjugate and preferably the label of the non-immobilised first specifically binding conjugate is the same kind as the label of the non-immobilised second specifically binding conjugate when the first- and second zones are not overlapping.

In the present context, the term “non-immobilised second specifically binding conjugate” refers to a conjugate which is freely movable in the moist state. Preferably the conjugate, is non-movable when it is dry but when moistened, the conjugate is released and starts migrating. The second specifically binding conjugate comprises a second molecule and a detectable label, the label being characterised as described above. In the present context, the term “second molecule” refers to the second member of the reference pair described above which together with a label provides the non-immobilised second specifically binding conjugate and is the part of the non-immobilised second specifically binding conjugate providing the specifically binding capacity.

In the present context the term “substantially non-movable” relates to the migration of the non-immobilised specifically binding molecule within the first zone when in the dry state. The migration of the non-immobilised specifically binding molecule within the first zone when in the dry state is less than 100% of the specifically binding molecules, such as less than 50%, e.g. less than 25%, such as less than 15%, e.g. less than 10%, such as less than 5%, e.g. less than 2%, such as less than 1%.

The migration of the non-immobilised specifically binding molecule out of the first zone when in the dry state is less than 100% of the specifically binding molecules, such as less than 50%, e.g. less than 25%, such as less than 15%, e.g. less than 10%, such as less than 5%, e.g. less than 2%, such as less than 1%. Preferably 0% of the non-immobilised specifically binding molecule migrates in the dry state.

In a preferred embodiment of the present invention, the molar ratio between the second molecule capable of binding to a compound different from the analyte to be assayed and the detectable label is in the range of 1:99 respectively, such as in the range of 25:75 respectively, e.g. in the range of 50:50, such as in the range of 75:25 respectively and e.g. in the range of 99:1 respectively.

Preferably, the device according to the present invention is provided with an application zone. In the present context, the term “application zone” refers to a site in the device where the liquid sample is applied to the device and which provides a fast absorption of the liquid sample and a fast and consistent release of sample to the first zone and to the porous zone. Accordingly, the material used in the application zone is selected from the group consisting of a nitrocellulose membrane, a polymer such as nylon, polyvinylidene fluoride or latex, glass fibre, woven fibres, non-woven fibres and a chromatographic gel membrane. Preferably the material used in the application zone is a woven or a non-woven glass fibre.

In a further preferred embodiment of the present invention, the application zone is part of the porous zone. In this embodiment the material used is the same as the material used in the porous zone.

In another preferred embodiment of the present invention, the application zone is separated from the porous zone. This separation gives the opportunity to provide the first zone with different characteristics than the porous zone, for instance different properties in the binding, different migration properties of the first specifically binding conjugate and the second specifically binding conjugate. In this embodiment, the material used can be the same as the material used in the porous zone or it can be different form the material used in the porous zone.

Preferably, the application zone is located upstream from the second and third zones and substantially upstream from first zone, and whereupon the liquid sample migrates through the first zone to release the non-immobilised first specifically binding conjugate and the non-immobilised second specifically binding conjugate. The application zone and the first zone can be partially overlapping. Preferably the two zones have about 1% overlap between the areas of the two zones, about 2% overlap between the areas of the two zones, about 3%, 4% 5%, 10%, 20%, 30%, 40%, 50%, 60%, 70%, 80%, 90% or about 100% overlap between the areas of the two zones is provided.

The material used in the application zone provides an appropriate bed volume relative to sample volume. In the present context, the term “appropriate bed volume relative to sample volume” relates to a bed volume large enough to receive the entire liquid sample applied without any of the liquid sample being lost. In a preferred embodiment of the present invention, the volume of the liquid sample to be applied to and absorbed by the application zone is preferably at most 1000 μl, such as at most 500 μl, for instance at most 200 μl, such as at most 175 μl, for instance at most 150 μl, such as at most 125 μl, for instance at most 100 μl, such as at most 75 μl, for instance at most 50 μl, such as at most 25 μl, for instance at most 10 μl.

The volume of the liquid sample to be applied to and absorbed by the application zone is preferably in the range of 1-1000 μl, such as in the range of 1-500 μl, for instance in the range of 1-200 μl, such as in the range of 1-175 μl, for instance in the range of 1-150 μl, such as in the range of 1-125 μl, for instance in the range of 1-100 μl, such as in the range of 1-75 μl, for instance in the range of 1-50 μl, such as in the range of 1-25 μl, for instance in the range of 1-10 μl, such as in the range of 10-200 μl, for instance in the range of 25-200 μl, such as in the range of 50-200 μl, for instance in the range of 100-200 μl.

The material used in the application zone comprises a large pore size to give a fast, consistent and quantitative uptake of the liquid sample. In a preferred embodiment of the present invention, the material used in the application zone is selected from a group of materials comprising a pore size preferably in the range of 10-30.000 μm, such as in the range of 10-20.000 μm, for instance in the range of 10-10.000 μm, for instance in the range of 10-1000 μm, such as in the range of 10-500 μm, such as in the range of 10-100 μm, for instance in the range of 10-75 μm, such as in the range of 10-50 μm, for instance in the range of 50-200 μm, such as the range of 50-100 μm, for instance in the range of 100-500 μm, such as the range of 50-300 μm, for instance in the range of 75-300 μm, such as the range of 75-200 μm, for instance in the range of 75-150 μm, such as the range of 75-120 μm.

In another preferred embodiment of the present invention, the materials used in the first zone and in the application zone provide low affinity for protein binding, molecular binding, peptide binding, immuno binding or hormone binding such as in the range of 5-400 μg/cm², for instance the range of 5-200 μg/cm², such as the range of 5-100 μg/cm², for instance the range of 5-50 μg/cm², such as the range of 5-40 μg/cm², for instance the range of 5-30 μg/cm², such as the range of 5-20 μg/cm², for instance the range of 5-10 μg/cm², such as the range of 10-20 μg/cm², for instance the range of 20-30 μg/cm², such as the range of 30-40 μg/cm² and for instance the range of 40-50 μg/cm², such as the range of 50-150 μg/cm² and for instance the range of 50-120 μg/cm², such as the range of 75-120 μg/cm² and for instance the range of 80-110 μg/cm². This reduces not only the non-specifically binding of the analyte to be assayed, the first specifically binding conjugate and the second specifically binding conjugate to the materials used but also reduces the non-specifically binding of other non-analyte compounds to be assayed.

In a preferred embodiment, the materials used in the first zone and in the application zone provide low retention of triglyceride rich samples. Additionally, this low retention of triglyceride rich samples can be accomplished, by applying at least one ancillary compound in the first zone and in the application zone.

Because of the complexity of the liquid samples to be assayed by the method of the present invention it may occasionally be an advantage to use an ancillary compound in order to improve the flow of the liquid sample through the zones and to provide a fast, consistent and quantitative release of the non-immobilised specifically binding molecule.

The ancillary compound may be supplied to the device either by a) adding it to the application zone alone or together with the liquid sample, b) incorporating the ancillary compound into at least one of the application zone, first zone, porous zone, second zone, third zone and/or adsorbent zone, or c) a combination thereof. The ancillary compound may also be added to the application zone before the liquid sample is added and the ancillary compound is maintained in a moistened state, or the ancillary compound and the liquid sample may be added to the application zone in layers. In the present context, the term “layers” refers to the splitting up of the volume of the ancillary compound and the volume of the liquid sample, whereupon the ancillary compound and the liquid sample is added to the application zone separately. In this case, the ancillary compound may be added as a liquid as well as a solid compound. The ancillary compound and the liquid sample may be split into at least 2 volumes each providing 4 alternating layers of ancillary compound and liquid sample, e.g. the ancillary compound and the liquid sample is split into at least 3 volumes each providing 6 alternating layers of ancillary compound and liquid sample, such as the ancillary compound and the liquid sample is split into at least 4 volumes each providing 8 alternating layers of ancillary compound and liquid sample, e.g. the ancillary compound and the liquid sample is split into at least 6 volumes each providing 12 alternating layers of ancillary compound and liquid sample, such as the ancillary compound and the liquid sample is split into at least 8 volumes each providing 16 alternating layers of ancillary compound and liquid sample, e.g. the ancillary compound and the liquid sample is split into at least 10 volumes each providing 20 alternating layers of ancillary compound and liquid sample, such as the ancillary compound and the liquid sample is split into at least 20 volumes each providing 40 alternating layers of ancillary compound and liquid sample.

In yet another embodiment of the present invention the the ancillary compound and the liquid sample is unevenly split resulting in 1-20 volumes of ancillary compound and/or 1-20 volumes of liquid sample which is provided to the application zone.

In a further embodiment of the present invention, the ancillary compound decreases non-specifically binding of the analyte and non specifically binding of the non-immobilised specifically binding molecule.

Additionally, the ancillary compound provides low affinity for unspecific protein binding.

In other embodiments of the present invention, the ancillary compound provides low retention of triglyceride rich samples and/or decreases the viscosity of the liquid sample.

In one embodiment of the present invention the ancillary compound contains chemical constituents selected from the group consisting of water, surfactant, salt, acid, base, metals, sugar, proteins, preservation agent and lipid.

In a further embodiment, improved flow of the liquid sample and fast, consistent and quantitative release of the non-immobilised specifically binding conjugate can be accomplished by applying an ancillary compound to at least one of the application zone, first zone, porous zone, second zone, third zone and/or adsorbent zone.

Additionally, the device according to the present invention is provided with an absorbent zone. In the present context, the term “absorbent zone” refers to a site located downstream from the second and third zones and is selected from the group of materials capable of increasing capillary effect in the porous zone, by providing force to the liquid sample to direct the migration from the application zone to the first zone and through the second and third zone to the end in the absorbent zone. The absorbent zone also provides a sponge effect by absorbing the liquid sample that has passed through the assay device.

Preferably, the material used in the absorbent zone is selected from a nitrocellulose membrane, a polymer such as nylon, polyvinylidene fluoride or latex, glass fibre, woven fibres, non-woven fibres and a chromatographic gel membrane.

The porous zone can be supported by a solid support. In the present context “solid support” refers to a material which has no influence on the migration or on the reaction of the liquid sample or on the specifically binding conjugates. The solid support provides a stabilising basis for the assay device and provides sufficient strength to maintain the desired physical shape and has substantially non interference with the production of a detectable signal. The material for the solid support may be selected from tubes, polymeric beads, nitrocellulose strips, membranes, filters and the like. Natural, synthetic, and naturally occurring materials that are synthetically modified, can be used as the material for the solid phase. Such materials include polysaccharides, for instance, cellulosic materials, such as paper and cellulosic derivatives, such as cellulose acetate and nitrocellulose, silica, inorganic materials, such as, for example, deactivated alumina, diatomaceous earth, MgSO₄ or other inorganic finely divided material uniformly dispersed in a porous polymeric matrix, wherein the matrix may comprise one or more polymers such as homopolymers and copolymers of vinyl chloride, for instance, polyvinyl chloride, vinyl chloride-propylene copolymer, and vinyl chloride-vinyl acetate copolymer, cloth, both naturally occurring (for instance, cotton) and synthetic (for instance, nylon), porous gels, such as silica gel, agarose, dextran, and gelatin, polymeric films, such as polyacrylamide, and the like.

In a presently preferred embodiment of the present invention, the material to be used as solid support is a pre-laminated card, such as Mylar backed or 3M Transperant Diagnostic Tape 9843R.

A device or a method based on the above principles can be used to determine a wide range of analytes by selecting appropriate specifically binding molecules and the invention is not limited to examples mentioned herein. The analytes to be assayed can be selected from proteins, haptens, immunoglobulins, antibodies, hormones, polynucleotides, steroids, drugs, and infectious disease agents such as bacteria.

In the present context, the term “steroid” refers to a group of chemical substances related in structure to one another and each containing the same kind of chemical skeleton comprising a tetracyclic cyclopenta[a]phenanthrene or a cyclopentanoperhydroxy-phenanthrene ring system or is a derivative compound of cholesterol. The group of chemical compounds to be assayed is selected from an adrenocorticoid, a progestin, an estrogen and an androgen. Non limiting examples of steroids to be assayed according to the present invention include pregnenolone, progesterone, testosterone, dihydrotestosterone, estrone, estradiol, cortisol, cortisone, aldosterone, corticosterone, androstenedione, 17α-OH-pregnenolone, 17α-OH-progesterone, 11-desoxy-corticosterone, 11-desoxycortisol and dehydroepiandrosterone.

In one presently preferred embodiment of the present invention, the steroid to be assayed is progesterone. Preferably progesterone is assayed in a liquid sample containing progesterone in concentrations of at most 100 ng/ml, at most 75 ng/ml, at most 50 ng/ml, for instance at most 30 ng/ml, such as at most 20 ng/ml, for instance at most 15 ng/ml, such as at most 10 ng/ml, for instance at most 5 ng/ml, such as at most 1 ng/ml, for instance at most 0.5 ng/ml, such as at most 0.1 ng/ml.

In another preferred embodiment of the present invention, the steroid to be assayed is estradiol. Preferably estradiol is assayed in a liquid sample containing estradiol in concentrations of at most 5 ng/ml, for instance at most 3 ng/ml, such as at most 2 ng/ml, for instance at most 1.5 ng/ml, such as at most 1 ng/ml, for instance at most 0.5 ng/ml, such as at most 0.1 ng/ml, for instance at most 0.05 ng/ml, such as at most 0.01 ng/ml.

In another preferred embodiment of the present invention, the steroid to be assayed is testosterone. Preferably, testosterone is assayed in a liquid sample containing testosterone in concentrations of at 20 ng/ml, for instance at most 10 ng/ml, such as at most 8 ng/ml, for instance at most 6 ng/ml, such as at most 5 ng/ml, for instance at most 4 ng/ml, such as at most 3 ng/ml, for instance at most 1 ng/ml, such as at most 0.5 ng/ml.

In a preferred embodiment of the present invention the analyte present in the sample is quantitatively determined without determining the amount of non-specifical binding in the second zone provided by the amount of second specifically binding conjugate bound in the second zone. Therefore, it is not required to measure non-specifical binding in the second zone provided by the amount of second specifically binding conjugate bound in the second zone for providing a highly sensitive assay. Thus, the assay is very easy to perform and provides a high reproducibility. In method various suitable labels for use in the present invention is selected from the group consisting of chromogens, catalysts, fluorescent compounds, chemiluminescent compounds, radioactive labels, metals, magnetic particles, dye particles, enzymes or substrates, or organic polymer latex particles; liposomes or other vesicles containing signal producing substances and the like.

The device according to the present invention may be applied for different purposes and used for measuring in varied types of samples including physiological fluids, organic fluids and naturally occurring fluids for detection and quantification of analytes.

A device according to the present invention may be used for measuring different physiological fluids. One system that can be used is a pregnancy test where the liquid sample used can be either blood or urine. Additionally, the device may be used for detecting diseases and infections for instance caused by microorganisms such as bacteria and yeast or other states of abnormal physiological states in a mammal. These physiological states can include the detection of analytes that are combined with compounds related to the consumption of illegal substances for improvement of for instance physical performance. Furthermore, the device may be used on the street for measuring the content of alcohol without taking blood samples, but by using for instance saliva.

The device of the present invention may be used in medical diagnostics for instance, in a laboratory, at the hospital, at the doctor or in an ambulance. The device can also be applied to measure different analytes in drinking water, waste water, sea water and ground water.

It is advantageous that the device of the present invention can be used without the use of additional reagents and the device can be used by a person not skilled in the art or by a person not having the professional skills, usually required in order to perform the analysis.

The device according to the present invention may also be applied in the analysis of food products, to control the content of for instance microorganisms, growth hormones and antibiotics.

The device can be compatible with automated or semi-automatic systems such as optimising performances of milk producing herds, when the assayed analyte is a steroid.

The following non-limiting embodiments and drawings will illustrate the invention further.

FIG. 1 shows the level of progesterone during the cow reproduction cycle. The solid line symbolises the level of progesterone in successfully inseminated cows and the dashed line symbolises the unsuccessfully inseminated cows.

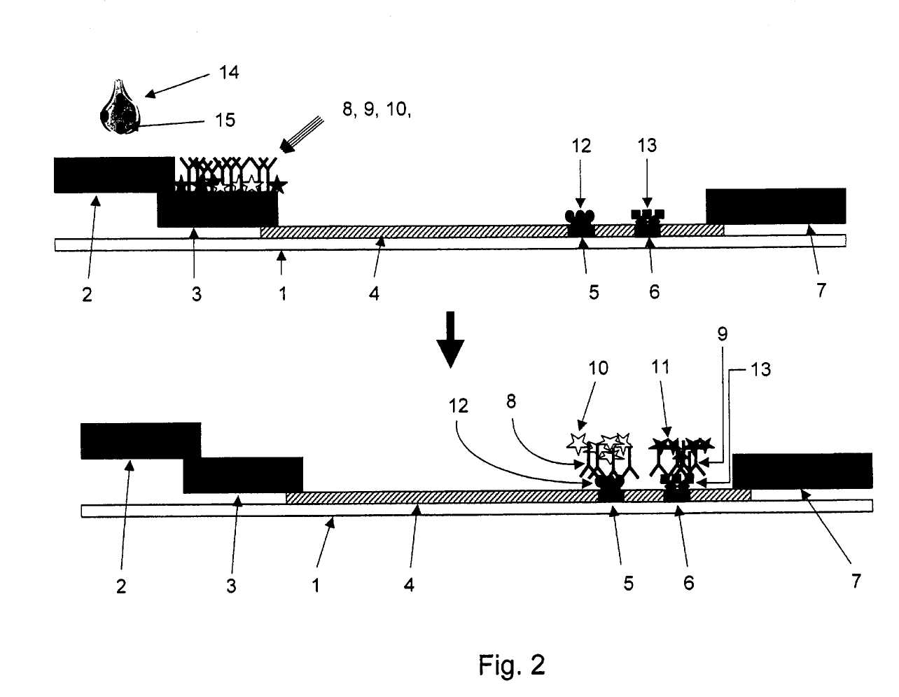

FIG. 2 shows an exemplary embodiment of the assay device according to the invention.

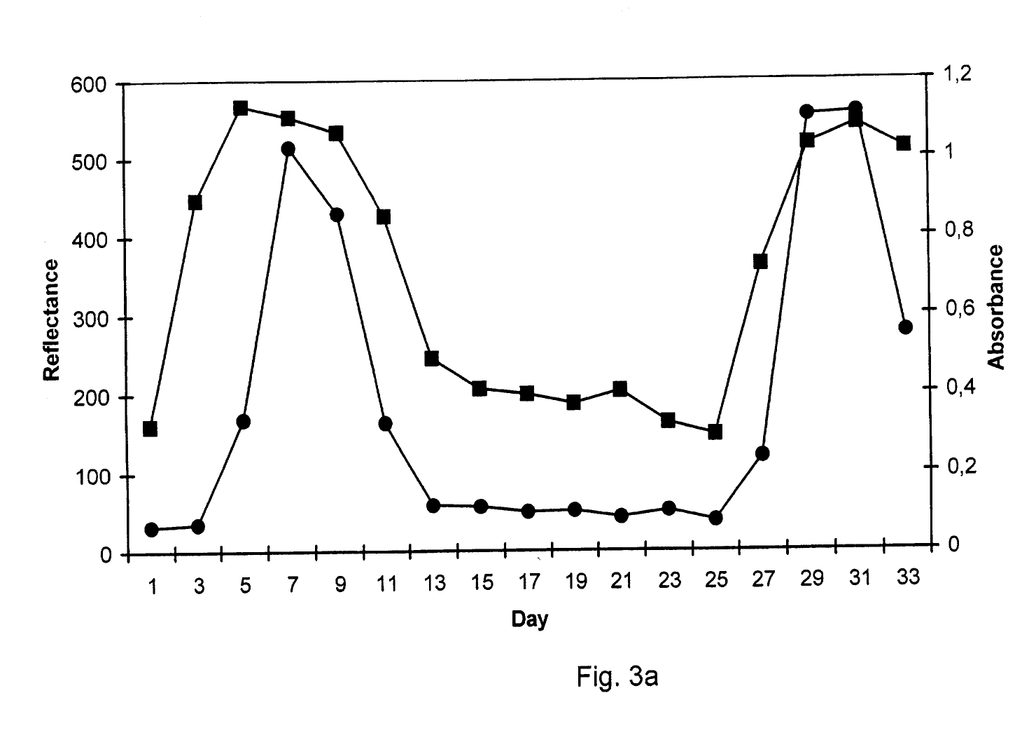

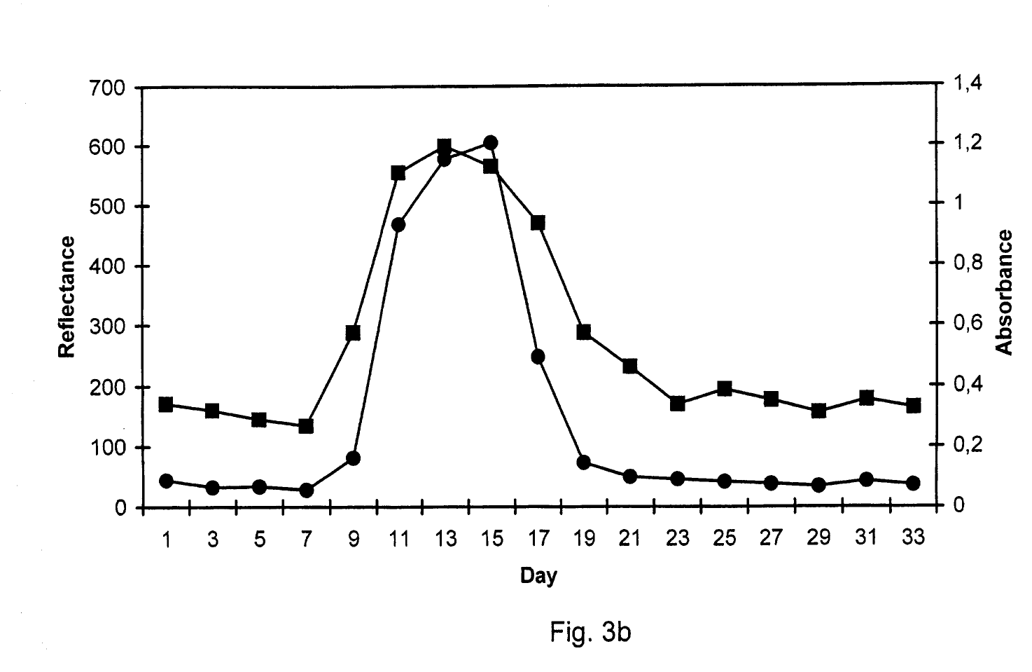

FIG. 3a shows assay during an oestrus cycle of raw milk samples collected from a cow after an unsuccessful insemination whereupon the cow was entered a new hormone cycle. Shown is raw data from the experiments performed with the wet chemical ELISA reference assay (squares) and the developed progesterone device of the present invention (circles), respectively. Thus, high values of absorbance/reflectance reflect low progesterone concentrations and vice versa.

FIG. 3b shows assay during an oestrus cycle of raw milk samples collected from a cow after a successful insemination followed by pregnancy. Shown are raw data from the experiments performed with the wet chemical ELISA reference assay (squares) and the developed progesterone device of the present invention (circles), respectively. Thus, high values of absorbance/reflectance reflect low progesterone concentrations and vice versa.

### DESCRIPTION OF THE PREFERRED EMBODIMENT

FIG. 2 represents two steps of the use of the immunoassay device according to the invention. First step is prior to sample application and the second step is post sample application and assay performance.

Referring to FIG. 2, the device comprises a solid support (1) having in one end an application zone (2) connected to a first zone (3) in which a first molecule (8) reactive to the analyte to be measured (15) and a second molecule (9) capable of binding specifically to a compound (13) different from the analyte to be assayed (15) are located. The first zone (3) is connected via a porous-zone (4) with an absorbent zone (7) in the other end of the solid support (1). The porous zone (4) comprises a second zone (5) and a third zone (6) and the third zone (6) is located downstream of the second zone (5). In the said second zone (5) an analyte (12) of the same type as the analyte to be assayed (15) is immobilised. In the said third zone (6) a compound (13) different from the analyte to be assayed (15) and which is capable of binding specifically to the second molecule (9) is immobilised. For providing detectable signals the first molecule (8) is coupled to a label (10) and provides a first specifically binding conjugate and this label (10) might be the same or be different from the label (11) coupled to the second molecule (9) which provides a second specifically binding conjugate.

In operation, the liquid sample (14) is added at the application zone (2), the liquid sample makes the application zone (2) and the first zone (3) wet and thereby releases the first specifically binding conjugate and the second specifically binding conjugate, found in the first zone (3). The first specifically binding conjugate consists of an analyte specific antibody (8) and a detectable label (10) for instance small gold particle, the second specifically binding conjugate consists of a compound (9) different from the analyte to be assayed (15) and a detectable label (11) for instance a small gold particles. While the liquid sample (14) and the first specifically binding conjugate and the second specifically binding conjugate migrate through the porous zone (4), immobilised analyte (12) in the second zone (5) of the same type as the analyte to be assayed (15) will bind the antibodies (8) which have not already been bound by the analyte (15) in the liquid sample (14) and the amount of detectable label (10) bound to the second zone (5) will be inversely proportional to the concentration of analyte (15) in the liquid sample (14).

Given the fact that the quality of the material of the porous zone (4) may be quite variable, resulting in a variable capillary flow rate and thereby a variable detectable signal at the second (5) and third zones (6), an internal reference system is included in the test strips. The internal reference system consists of a second molecules (9) such as antibodies conjugated to for instance gold particles (11) which are also provided in the first zone (3) and a compound (13) specific for the said antibodies (9) are immobilised in the third zone (6) and the test will be read by measuring a ratio of the signals at the second zone (5) and at the third zone (6).

It will be seen from the above description that there are many variations that can be made in respect of reagents, materials, analytes, rearrangements of zones and the like, all without leaving the scope of the present invention.

### EXAMPLES

### Example 1

The purpose of this experiment is to illustrate that the device of the present invention is able to measure very small variations in progesterone concentrations quantitatively in milk samples collected during an oestrus cycle and that it is possible to measure small variations in the progesterone concentrations with a high reproducibility. Thus, to show it is possible with the developed device to identify heat and pregnancy/non-pregnancy

### Method

20 μl of milk and 100 μl of ancillary compound are added simultaneously to the first zone of the progesterone dry sticks. The sticks are incubated 5 min at room temperature and subsequently read in a designated reader measuring reflectance at 420 nm.

### RESULTS

Obtained results are shown in FIG. 3a and 3b where the adsorbance and reflectance measured using the ELISA reference assay and the developed progesterone assay is plotted against day of collecting the milk sample. As it can be seen, the device is capable of providing quantitative measurements of progesterone similar to what could be obtained using the ELISA assay. Also, it was possible with the device of the invention to identify heat and to differentiate whether the insemination of the cow is successful (FIG. 3b) or unsuccessful (FIG. 3a).

1. A device for assaying an analyte in a liquid sample, said device comprising a porous zone permitting the migration of the sample, said porous zone comprising
(i) a first zone comprising a non-immobilised first specifically binding conjugate consisting of a first molecule capable of specifically binding to the analyte to be assayed and a detectable label, and a non-immobilised second specifically binding conjugate consisting of a second molecule capable of binding specifically to a compound different from the analyte to be assayed and a detectable label, said second specifically binding conjugate is not capable of specifically binding to the analyte to be assayed,
(ii) a second zone comprising the same type of analyte as the one to be assayed or an analogue thereof, said same type of analyte or analogue being capable of specifically binding to the first specifically binding conjugate, the analyte or analogue being immobilised to the porous zone, and
(iii) a third zone comprising immobilised thereto said compound different from the analyte to be assayed that is capable of binding specifically to the second specifically binding conjugate.

2. A device according to claim 1 where the label of the non-immobilised first specifically binding conjugate and/or the label of the non-immobilised second specifically binding conjugate is a metal

3. A device according to claim 2 where the metal is selected from the group consisting of gold, silver and titanium.

4. A device according to any of claims 1-3 where the label of the non-immobilised second specifically binding conjugate is of the same type as the label of the first specifically binding conjugate.

5. A device according to any of claims 1-3 where the second and third zones are at least partially overlapping.

6. A device according to any of claims 1-5 where the same type of analyte as the one to be assayed or an analogue thereof in the second zone and/or the compound in the third zone that is different from the analyte to be assayed is coupled to the porous zone via a spacer molecule.

7. A device according to claim 6 where the spacer molecule is a peptide.

8. A device according to any of claims 1-7 further comprising a sample application zone.

9. A device according to claim 8 where the first zone and the application zone are separated.

10. A device according to any of claims 1-9 further comprising an absorbent zone.

11. A device according to any of claims 1-10 further comprising a solid support.

12. A device according to any of claims 1-11 where the first molecule in the first zone is selected from the group consisting of antibodies and receptors.

13. A device according to any of claims 1-12 wherein the analyte to be assayed is a steroid selected from the group consisting of a progestagen, an estrogen and an androgen.

14. A device according to claim 13 where the progestagen to be assayed is progesterone.

15. A device according to claim 14 permitting the assaying of progesterone in a liquid sample containing 0-50 ng/ml of progesterone.

16. A device according to any of claims 1-15 that comprises two or more first and second zones permitting the simultaneous assaying of two or more analytes.

17. An appliance carrying a multiplicity of the device according to any of claims 1-16.

18. An appliance according to claim 17 where an automatic, a semi-automatic and a continuous system is provided.

19. An appliance according to any of claims 17 or 18 where the appliance is a strip.

20. A method for assaying an analyte in a liquid sample, comprising the steps of:
(i) permitting migration from a first zone through a porous zone of the liquid sample and a non-immobilised first specifically binding conjugate consisting of a first molecule capable of specifically binding to the analyte to be assayed and a detectable label, and a non-immobilised second specifically binding conjugate consisting of a second molecule capable of binding specifically to a compound different from the analyte to be assayed and a detectable label, said second specifically binding conjugate is not capable of specifically binding to the analyte to be assayed nor to the first specifically binding conjugate,
(ii) permitting non-specifically bound first specifically binding conjugate to bind in a second zone comprising the same type of analyte as the one to be assayed or an analogue thereof, said same type of analyte or analogue being capable of specifically binding to the first specifically binding conjugate, the analyte or analogue being immobilised to the porous zone, and
(iii) permitting second specifically binding conjugate to bind in a third zone comprising immobilised thereto said compound different from the analyte to be assayed that is capable of binding specifically to the second specifically binding conjugate.

21. A method according to claim 20 wherein the analyte present in the sample is determined quantitatively without determining the amount of non-specifical binding in the second zone provided by the amount of second specifically binding conjugate bound in the second zone.

22. A method according to any of claims 20-21 wherein the analyte to be assayed is selected from the group consisting of a progestagen, an estrogen and an androgen.

23. A method according to claim 22 wherein the progestagen to be assayed is progesterone.

24. A method according to claim 23 permitting that assaying of progesterone in a sample containing 0-50 ng/ml hereof.

25. A method according to any of claims 20-24 wherein the first specifically binding conjugate is selected from the group consisting of antibodies and receptors.

26. A method according to claim 25 wherein the antibodies are monoclonal antibodies.

27. A method according to any of claims 20-26 wherein a reference pair is selected from the group providing a specifically binding consisting of a protein-antibody binding, an antigen-antibody binding, an antibody-antibody binding, a lectin-carbohydrate binding, a hormone-antibody binding and a hormone-receptor binding.

28. A method according to any of claims 20-27 wherein the porous zone comprise of a porous material, said porous material is selected from the group consisting of a nitrocellulose membrane, a polymer such as nylon, polyvinylidene fluoride or latex, glass fibre, woven fibres, non-woven fibres and a chromatographic gel membrane.

29. A method according to claim 28 wherein the average pore size of the porous material is in the range of 10-10.000 nm.

30. A method according to any of claims 20-28 wherein the capacity of the porous material to bind proteins is in the range of 1-400 μg/cm².

31. A method according to any of claims 20-30 wherein the capillary flow-rate of the porous material is in the range of 50-250 sec/4 cm.

32. A method according to any of claims 20-31 wherein the first zone, an application zone and an adsorption zone uses the same type of material is used as the porous zone.

33. A method according to any of claims 20-32 wherein the material used in the first zone provides a fast, consistent and quantitative release of non-immobilised first specifically binding conjugate and non-immobilised second specifically binding conjugate.

34. A method according to any of claims 32-33 wherein the materials used in the first zone and in the application zone provide low affinity for protein binding.

35. A method according to any of claims 32-34 wherein the materials used in the first zone and in the application zone provide low retention of triglyceride rich samples.

36. A method according to any of claims 32-34 wherein the retention of triglyceride rich samples are reduced by applying at least one ancillary compound in the first zone and in the application zone.

37. A method according to any of claims 20-36 wherein the detectable label is selected from the group consisting of dyes, enzymes, fluorescent compounds, chemiluminescent compounds, radioactive labels and metals.

38. A method according to claim 37 wherein the detectable label is selected from the group consisting of gold, silver, carbon, fluorescent latex beads and dyed latex beads.

39. A method according to any of claims 20-38 wherein the assay time is less than 15 min.

40. A method according to any of claims 20-39 wherein the liquid sample to be assayed is mammalian physiological fluids.

41. A method according to claim 40, wherein the mammalian physiological fluids to be tested is selected from a group consisting of milk samples, urinary samples, blood samples and saliva samples.

42. A method according to any of claims 40-41 wherein the mammal is a cow or a human.

43. A method according to any of the claims 20-42, wherein a device as described in the claims 1-14 and an appliance as described in the claims 15-17 are used.
